SEQUENCE LISTING (14 sequences)

SEQ ID NO: 1 - DNA, 28 bp, Artificial Sequence
  Proximal NFkB binding regions of the murine iNOS promoter
ccaactggggactctccctttgggaaca

SEQ ID NO: 2 - DNA, 27 bp, Artificial Sequence
  Sense primer N1
tgcgaggccagaggccacttgtgtagc

SEQ ID NO: 3 - DNA, 24 bp, Artificial Sequence
  Antisense primer A1
tgtagccctggctgttctgtactc

SEQ ID NO: 4 - DNA, 24 bp, Artificial Sequence
  Sense primer S1
cgactccaccaacacttacacaag

SEQ ID NO: 5 - DNA, 24 bp, Artificial Sequence
  Antisense primer A1
tgtagccctggctgttctgtactc

SEQ ID NO: 6 - DNA, 20 bp, Artificial Sequence
  Target sequence for SMS2 siRNA
ccgtcatgatcacagttgta

SEQ ID NO: 7 - DNA, 21 bp, Artificial Sequence
  Scrambled siRNA target sequence
gacgacggagtgtgttaatta

SEQ ID NO: 8 - DNA, 20 bp, Artificial Sequence
  Forward
ggttcccacagaaaccaaga

SEQ ID NO: 9 - DNA, 20 bp, Artificial Sequence
  Reverse
gatgcctgttttccaccact

SEQ ID NO: 10 - DNA, 21 bp, Artificial Sequence
  Forward primer sequence for 18S rRNA
agtccctgccctttgtacaca

SEQ ID NO: 11 - DNA, 21 bp, Artificial Sequence
  Reverse primer sequence for 18S rRNA
gatccgagggcctcactaaac

SEQ ID NO: 12 - DNA, 24 bp, Artificial Sequence
  Probe sequence
cgcccgtcgctactaccgattggt

SEQ ID NO: 13 - DNA, 20 bp, Artificial Sequence
  Forward primer sequence
gtcttgcaagctgatggtca

SEQ ID NO: 14 - DNA, 20 bp, Artificial Sequence
  Reverse primer sequence
accactcgtacttgggatgc

(19) United States
(12) Patent Application Publication
(10) Pub. No.: US 2009/0264514 A1
(43) Pub. Date: Oct. 22, 2009

(54) SPHINGOMYELIN SYNTHASE 2 (SMS2) DEFICIENCY ATTENUATES NFkB ACTIVATION, A POTENTIAL ANTI-ATHEROGENIC PROPERTY

(75) Inventors: Xian-Cheng Jiang, Fort Lee, NJ (US); Tiruneh K. Hallemariam, Forest Hills, NY (US)

Correspondence Address: HOFFMANN & BARON, LLP, 6900 JERICHO TURNPIKE, SYOSSET, NY, 11791, US

(73) Assignee: The Research Foundation of State University of New York, Albany, NY (US)

(21) Appl. No.: 12/426,324

(22) Filed: Apr. 20, 2009

Related U.S. Application Data
(60) Provisional application No. 61/046,240, filed on Apr. 18, 2008.

(51) Int. Cl. A61K 31/7105; C12Q 1/02; C12N 9/99

(52) U.S. Cl. 514/44 R; 435/29; 435/184

(57) ABSTRACT

The present invention is directed to a method of screening for NFκB inhibiting agents, the method including the steps of administering a biologically effective amount of a candidate SMS2 inhibitor to at least one cell; and determining whether the candidate SMS2 inhibitor inhibits NFκB.

### CROSS REFERENCE TO RELATED APPLICATIONS

The present application claims the benefit of and priority to U.S. Patent Application No. 61/046,024, filed on Apr. 18, 2008, the contents of which is incorporated by reference herein in its entirety.

### FUNDING STATEMENT

This invention was made with government support under contract identifier HL-69817 and HL-64735 from the National Institute of Heath and by contract identifier Grant-in-Aid 0755922T from the American Heart Association. The government has certain rights to the invention.

### FIELD OF THE INVENTION

The present invention relates to the discovery that a sphingomyelin synthase isotope, SMS2, deficiency decreases plasma membrane sphingomyelin levels and thus attenuates NFκB activation. Specifically, the present invention includes a method of screening SMS inhibitors and methods of treating atherosclerosis.

### RELATED ART

Atherosclerosis and its associated coronary artery disease (CAD) is the leading cause of mortality in the industrialized world. However, no wholly satisfactory lipid-modulating therapies exist. Some lipid modulating therapies have tolerance issues, while other have limited effectiveness. As a result, there is a significant unmet medical need for a well-tolerated agent, which can lower plasma LDL levels and/or elevate plasma HDL levels (i.e., improving the patient's plasma lipid profile), thereby reversing or slowing the progression of atherosclerosis.

Although there are a variety of anti-atherosclerosis therapies, there is a continuing need and a continuing search for alternative therapies for the treatment of atherosclerosis and dyslipidemia.

### SUMMARY OF THE INVENTION

An aspect of the present invention provides a method of screening for NFκB inhibiting agents. The method of screening NFκB inhibiting agents includes administering a biologically effective amount of a candidate SMS2 inhibitor to at least one cell; and determining whether the candidate SMS2 inhibitor inhibits NFκB.

Another aspect of the present invention provides a method for attenuating inflammation induced by NF-kB, including inhibiting sphingomyelin synthase (SMS2) in the plasma membrane of at least one cell.

Still another aspect of the present invention provides a method of regulating an NFκB activation which includes modulating an SMS2 in at least one cell.

These and other features of the invention will be better understood through a study of the following detailed description and accompanying drawings.

### BRIEF DESCRIPTION OF DRAWINGS

FIG. 1 The impact of SMS2 KO (knockout) and SMS2 siRNA on SMS2 mRNA, total cellular sphingomyelin synthase (SMS) activity, de novo Sphingomyelin (SM) synthesis, and plasma membrane SM levels. FIG. 1A, RT-PCR analysis of SMS2 mRNA using total RNA extracted from (wild type) WT and SMS2 KO macrophages. FIG. 1B, SMS2 mRNA levels were determined by real-time PCR in HEK293 cells after 24 hr of siRNA transfection. FIG. 1C, SMS activity in mouse macrophages was conducted using total cell lysate. FIG. 1D. SMS activity 48 hr after SMS2 siRNA transfection in HEK293 cells using total cell lysates. Value are mean±SD, N=5, *P<0.05. FIG. 1E, de novo SM biosynthesis in macrophages. FIG. 1F, HEK 293 cell de novo SM biosynthesis. FIG. 1G, Lysenin sensitivity of macrophages. FIG. 1H. Lysenin sensitivity of HEK 293 cells. Assay was conducted as in macrophages. Values are mean±SD, expressed as percentage of control, N=5, *P<0.01.

FIG. 2 Activation and nuclear translocation of NFκB (Nuclear Factor Kappa B). Macrophages were stimulated with LPS and HEK 293 cells were stimulated with TNFα (alpha) for the indicated durations and their nuclear and cytoplasmic extracts were probed with anti-p65 and anti-IκBα (anti-I kappa B alpha) antibody, respectively. Anti-histone 3 (H3) and anti-GAPDH antibodies were used as nuclear and cytoplasmic protein loading controls, respectively. FIG. 2A, Nuclear NFκB in control vs SMSKO macrophages. FIG. 2B, IκBα levels in control vs SMSKO macrophages. FIG. 2C, Nuclear NFκB in control vs SMS2 siRNA transfected HEK293 cells. FIG. 2D, IκBα levels in control vs SMS2 siRNA transfected HEK293 cells. FIG. 2E and FIG. 2F, Immunocytochemistry of NFκB. E, Macrophages stimulated with LPS (200 ng/ml) for 30 minutes. FIG. 2F, HEK 293 cells stimulated with 20 ng/ml TNFα for 20 minutes. These results are a representative of three independent experiments. It should be noted that FIG. 2E and FIG. 2F are not on the same scale.

FIG. 3 SMS2 deficiency influences transcriptional activity of NFκB. FIG. 3A, Reporter gene assay in HEK293 cells. Cells were sequentially transfected with siRNA and 500 ng/ml κB-luciferase and 25 ng/ml renilla construct. Twenty four hours later, the cells were harvested and lysated. The assay was conducted according to the manufacturers protocol (Promega). FIG. 3B, mRNA levels of iNOS were determined for control and SMS2 KO macrophages by real-time PCR after LPS (200 ng/ml) treatment for the indicated durations. FIG. 3C, iNOS protein levels for wild type and SMS2KO macrophages treated with LPS (200 ng/ml) and IFNγ (20 ng/ml) for the indicated time. FIG. 3D and FIG. 3E, EMSA assay for mouse iNOS promoter fragment (probe) binding ability of NFκB in wild type and SMS2 KO macrophages. FIG. 3D, nuclear extracts of WT macrophage after LPS stimulation were used to optimize the EMSA system. Antibodies to the p65 and p50 subunits of NFκB were used for supershift assay. Anti-p300, -C-rel, and -Mitf antibodies were used as negative controls. 1. probe; 2. Nuclear extracts; 3. Nuclear extracts+50 fold cold probe; 4. Nuclear extracts+anti-p50 Ab; 5. Nuclear extracts+anti-p65 Ab; 6. Nuclear extracts+anti-c-Rel; 7. Nuclear extracts+anti-p300 Ab; and 8. Nuclear extracts+anti-Mitf Ab. FIG. 3E, Comparison of NFκB binding to the iNOS promoter for control vs SMS2 KO macrophages. Results shown are representative of three independent experiments. Values are mean±SD, *P<0.01.

FIG. 4 Recruitment of TNFR1 to lipid rafts in HEK293 cells. For raft isolation, cells were homogenized at 4 degrees C. with lysis buffer containing 1% Triton X-100. Fractions were obtained after discontinuous sucrose gradient centrifugation. Equal aliquots of fractions were subjected to SDS-PAGE, and proteins were probed by western blotting. Raft fractions were identified by the enrichment of the raft marker lyn and absence of the non-raft resident CD71, transferrin receptor. FIG. 4A. Comparison of TNFR1 in fractions in non-stimulated or 5 minute TNFα stimulated HEK293 cells transfected with SMS2 or control siRNA. FIG. 4B. Representative fractions of rafts or non rafts were compared after 0, 5 and 15 minutes of TNFα stimulation. FIG. 4C. Western blots of whole cell lysate using specific antibodies for TNFR1, and GAPDH. Results shown are a representative of three independent experiments.

FIG. 5 Internalization of the TNFα-TNFR1 complex in HEK 293 cells and plasma membrane recruitment of TLR4-MD2 in macrophages. FIG. 5A, FACS analysis of cell surface TNFR1 using phycoerythrin conjugated anti-TNFR1 antibody in control (blue) and SMS2 siRNA (green) transfected HEK293 cells. Results shown are a representative of three independent experiments. FIG. 5B, Specific binding of [¹²⁵I]-TNFα to cell surface TNFR1 at 4 degrees C. Values are mean±SD, N=4, *P<0.001. FIG. 5C, Internalization of [¹²⁵I]-TNFα-TNFR1 complex at 37 degrees C. Values are mean±SD, N=4, *P<0.001. FIG. 5D. Macrophages were stained with 1 microg/mL TLR4-MD-2 complex antibody for 1 hr on ice, then washed with ice cold PBS for 3 times before analyzed on a FACScan with CellQuest software. Results shown are a representative of three independent experiments.

FIG. 6 Strategy used to disrupt the mouse SMS2 gene. FIG. 6A, The bottom line represents the map of the endogenous mouse SMS2 gene and its flanking sequence. The top line shows the predicted organization of the locus after homologous recombination. A pair of PCR primers indicated was used to confirm the integrity of site-specific integration. FIG. 6B, Tail tip DNA was extracted. Genomic PCR was performed. Wild type mouse (+/+) DNA shows a 760 bp band; heterozygous knockout mouse (+/−) DNA shows 760 bp and 970 bp bands; and homozygous knockout mouse (−/−) DNA shows a 970 bp band. NE, neomycin-resistant gene; WT, wild type; KO, knockout.

FIG. 7 Western blot of NFκB nuclear translocation and IkBα degradation in HEK293 cells transfected with SMS siRNA. Cells were stimulated with 20 ng/ml TNFα for different durations 48 hr after siRNA transfection. Cells were lysed and the cytoplasmic and nuclear fractions isolated. FIG. 7A; Western blot was conducted using the nuclear fraction with anti NF-kB (p65) antibody. FIG. 7B; Western blot was conducted using the cytoplasmic fraction with anti IkBα antibody. NT, no treatment.

FIG. 8 Immunocytochemistry of NFκB in HEK293 cells. SMS1 and control siRNA transfected cells were stimulated with TNFα for 10 minutes and washed, permeabilized and incubated with anti-NFκB antibody and fluorescent conjugated secondary antibody. Cells were then mounted with a solution containing DAPI, for nuclear staining, and then visualized with a fluorescent microscope.

FIG. 9 SMS1 siRNAs decrease TNFα-stimulated NFκB reporter gene expression in HEK293 cells. HEK293 cells were simultaneously transfected with siRNA and 500 ng/ml κB-luciferase and 25 ng/ml renilla construct. Twenty four hours later, the cells were treated with 20 ng/ml of TNFα for 8 hr, then harvested and lysated. The supernatant was used in the dual luciferase assay system according to the manufacturers protocol (Promega). Relative luciferase values were standardized with the renilla control. Results shown are representative of two independent experiments. Values are mean±SD, N=5, *P<0.001.

FIG. 10 SMS2 deficiency attenuate MAP kinase activity. After LPS treatment, macrophages from SMS2 and WT mice were homogenized. Equal aliquots were subjected to SDS-PAGE, and proteins and phosphoproteins were probed by western blotting using a MAPK Family Antibody Samples Kit (Cell Signaling).

### DETAILED DESCRIPTION OF THE INVENTION

Atherosclerosis is an inflammatory disease. The accumulation of macrophage-derived foam cells in the vessel wall is always accompanied by the production of a wide range of chemokines, cytokines, and growth factors.¹ These factors regulate the turnover and differentiation of immigrating and resident cells, eventually influencing plaque development. One of the key regulators of inflammation is NFκB,² which has long been regarded as a proatherogenic factor, mainly because of its regulation of many of the proinflammatory genes linked to atherosclerosis.^{3,4}

Sphingomyelin (SM) is one of the major lipids on the plasma membrane and is enriched in lipid rafts, which are considered microdomains of plasma membrane critical for signal transduction.^{5,6} The inventors herein found that the depletion of cholesterol from rafts causes a redistribution of TNFα receptor 1 to non-raft plasma membrane, preventing NFκB activation⁷ or ligand-induced RhoA activation,⁸ and such treatment also inhibits proinflammatory signals mediated by TLRs.⁹ Studies also suggest that NfκB activation is triggered by SM-derived ceramide.^{10,11} On the contrary, it has been shown that ceramide is not necessary or even inhibits NfκB activation.¹²

SM biosynthesis might also affect NFκB activation. SM is synthesized by sphingomyelin synthase (SMS), which transfers the phosphorylcholine moiety from phosphatidylcholine (PC) onto ceramide, producing SM and diacylglycerol (DAG).¹³ Lumberto et al.¹⁴ found that D609, a nonspecific SMS inhibitor, blocks TNFα- and phorbol ester-mediated NFκB activation that was concomitant with decreased levels of SM and DAG. This did not affect the generation of ceramide, suggesting SM and DAG derived from SM synthesis are involved in NFκB activation. However, D609 is widely used to inhibit PC-phospholipase C (PC-PLC), a well-known regulator of NFκB activation via DAG signaling.¹⁵ Thus it remains unclear which pathway D609 inhibits to cause a diminished NFκB activation

Two SMS genes, SMS1 and SMS2, have been cloned and characterized for their cellular localizations^{16,17} SMS1 is found in the trans-golgi apparatus, while SMS2 is predominantly found at the plasma membrane.¹⁶ The present inventors and other investigators have shown that SMS1 and SMS2 expression positively correlate with levels of SM in lipid rafts.¹⁸⁻²⁰ Furthermore, SMS1 has been implicated in the regulation of lipid raft SM level and raft functions such as FAS receptor clustering,¹⁸ endocytosis, and apoptosis.¹⁹ However, the role of SMS2, the major SMS on the plasma membrane, in cell signaling, including NFκB activation, is unknown.

The role of SMS2 in NFκB activation was studied by utilizing SMS2 KO mouse macrophages and SMS2 siRNA-treated HEK293 cells. In both cells, it was unexpectedly discovery that SMS2 deficiency significantly attenuates NFκB activation. Thus, SMS2 is a modulator of NFκB activation, and may play important roles in inflammation during atherogenesis.

The present inventors have shown a novel and essential role of SMS2 in modulating NFκB activation with their experiments. This is based on the following observations: in both SMS2 KO mouse macrophages and SMS2 knockdown HEK293 cells, 1) SMS activity, de novo SM synthesis, cellular and plasma membrane SM levels were significantly decreased, 2) ligand-induced NFκB activation, including IκBα degradation and NFκB nuclear translocation, as well as transcriptional activation, were significantly attenuated, and 3) LPS-induced membrane recruitment of TLR4-MD2 complex and TNFα-induced raft association of TNFR1 were impaired in SMS2 KO macrophages and SMS2 siRNA treated HEK293 cells, respectively.

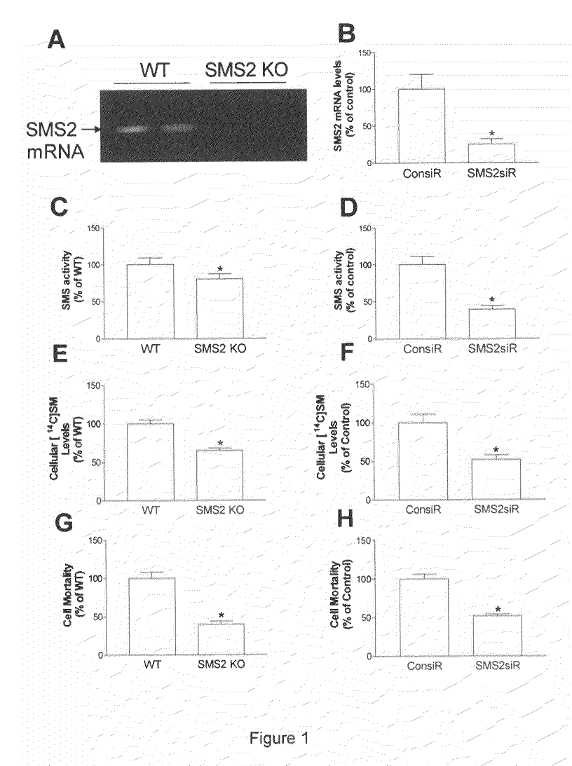

SMS2 makes an important contribution to the de novo SM biosynthesis and total cellular SM levels. Based on their relative proximity to the site of ceramide biosynthesis, it has been suggested that SMS1 might be involved in the de novo SM biosynthesis while SMS2 is involved in the remodeling of plasma membrane structure.²⁸ However, in the study results published by the present inventors which is incorporated herein by reference in its entirety, SMS2 was found to participate in de novo SM biosynthesis (FIGS. 1E and 1F). (²⁰, Li, Z., et al, Inhibition of sphingomyelin synthase (SMS) affects intracellular sphingomyelin accumulation and plasma membrane lipid organization. Biochim. Biophysi. Acta. 2007, Volume 9, September 2007; 1771:1186-1194.)

In support of the experiments and the present invention, a recent report indicated that both SMS1 and SMS2 are required for SM homeostasis and growth in human HeLa cells.²⁹ SMS1 and SMS2 are co-expressed in a variety of cells with different ratios, suggesting that the genes contribute variably to cellular SM depending on the cell type. Intriguingly, in some cells, such as Huh 7 cells and macrophages, SMS2 contributes only 20% of the total SMS activity measured in vitro, whereas, SMS2 depletion disproportionately reduces cellular SM levels (Table 1). This suggests that, in vivo, SMS1 and SMS2 activities depend on their local environments, such as availability of substrates.

SM synthesis by SMS2 is important for maintaining plasma membrane structure. Previously, the present inventors found that knockdown of SMS2 caused a depletion of SM in membrane lipid rafts.²⁰ The present work of the inventors supports these observations, and shows that intact SMS2 KO macrophages (FIG. 1G) and SMS2 siRNA treated HEK293 cells (FIG. 1H) have a stronger resistance to lysenin-mediated lysis than that of controls. The results suggest the physiological role of SMS2 in the formation and/or maintenance of SM-enriched lipid microdomains or lipid rafts on the plasma membrane. Consistent with the observations of the present inventors, studies of SMS2 function in sperm cell also suggest that SMS2 is important for reconstruction of plasma membrane structure.³⁰

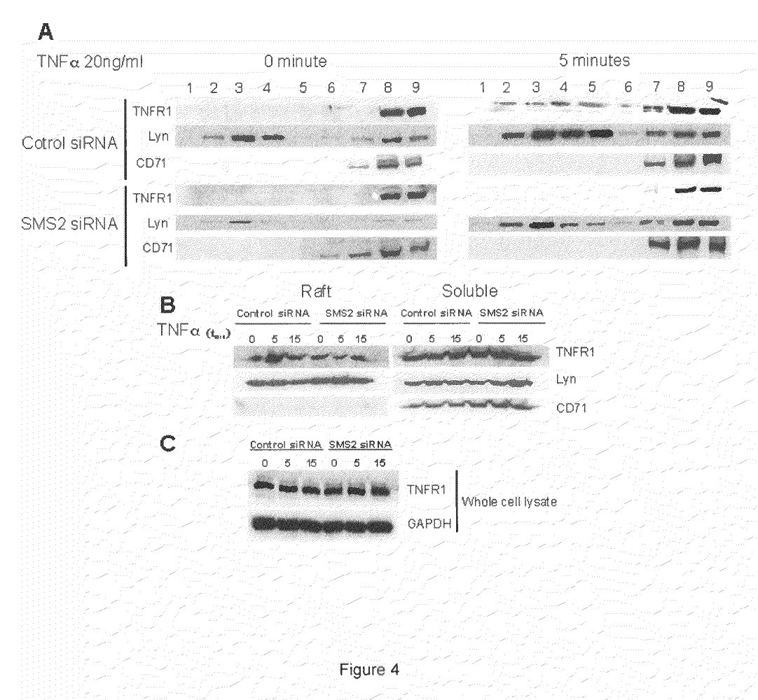

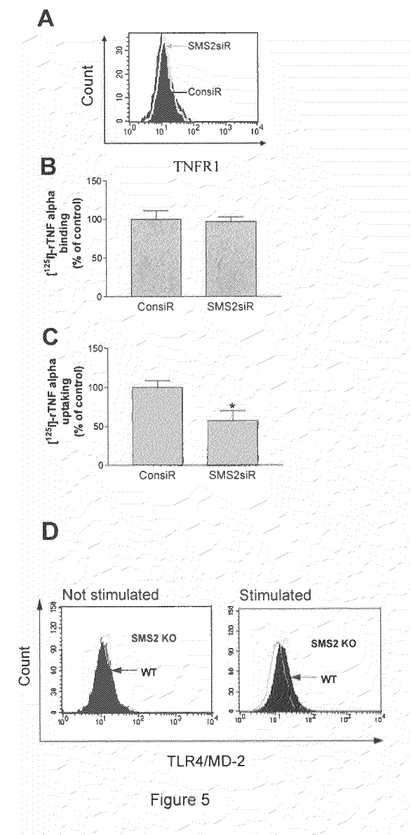

SMS2 deficiency could alter signal transduction mediated by lipid raft-associated receptors. As reported, the interaction of SM and cholesterol drives the formation of plasma membrane rafts,⁵ and the relative proportions of both SM and cholesterol appear critical for the stability and function of lipid rafts.^{5,18,19} In the present study, it was found that upon stimulation by TNFα, the recruitment of TNFR1 receptor to lipid rafts following ligand stimulation was blocked in SMS2 knockdown cells (FIG. 4B) suggesting a mechanism for the modulation of NFκB activity by SMS2. This finding is in agreement with previous reports where raft association of TNFR1 found to be crucial for TNFα-mediated NFκB activation in human fibrosarcoma cells.⁷ Similar to earlier report that the activity of SMS1 is required for effective raft mediated endocytosis,¹⁹ the present inventors found that SMS2 knockdown also reduced ligand-induced internalization of the TNFR1 receptor (FIG. 5C). Also, it was found that LPS-induced plasma membrane recruitment of TLR4-MD-2 complex was diminished in SMS2 KO macrophages (FIG. 5D). Taken together, these findings strongly suggest the critical role of SMS2 synthesized SM for the normal function of TNFR1 and TLR4 receptors on the plasma membrane following stimulation by their respective ligands.

Luberto et al.¹⁴ indicated that, in the absence of SMS activity cellular ceramide inhibits NFκB activation, but under high SMS, the resulting DAG signal stimulates NFκB. Here, the present inventors demonstrated that SMS2 deficiency shifts the cellular ceramide and DAG balance in favor of ceramide (Table 1). Cellular DAG functions as activator of both conventional and novel protein kinase C,³¹⁻³², a family of serine/threonine kinases that regulate a diverse set of cellular processes, including NFκB activation.^{33,34} Several pathways can lead to the generation of DAG.³¹ Due to the absence of specific SMS inhibitor, whether the DAG generated by SMS regulates cellular functions is unknown. In this study, in line with a decreased activity of NFκB, direct evidence is provided for a significant reduction in macrophage DAG levels as a consequence of SMS2 deficiency. The absence of the reduction of DAG level in SMS2 knockdown HEK293 cells may reflect the intrinsic difference between these cell type and mouse macrophages.

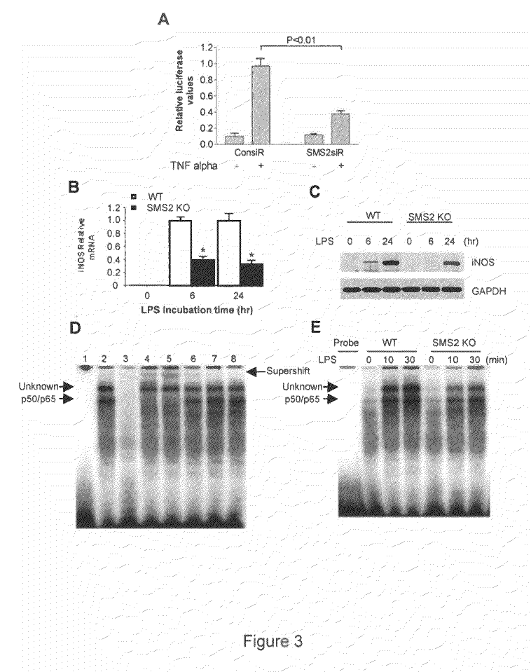

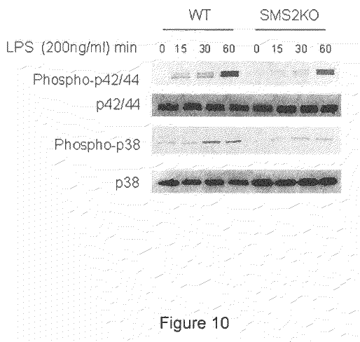

SMS2 deficiency may also influence signal transduction pathways other than NFκB activation. The activation of MAP kinases was attenuated in SMS2 KO macrophages (FIG. 10). Moreover, in the EMSA analysis, in addition to NFκB, an unknown shifted complex was noted (FIGS. 3D and 3E). This unknown complex could not be supershifted by any of the anti-NFκB (p50/p65), or with antibodies against the other NFκB family proteins C-Rel and p300 (FIG. 3D). The identification of this complex and its relationship to SMS2 and NFκB warrant further investigation.

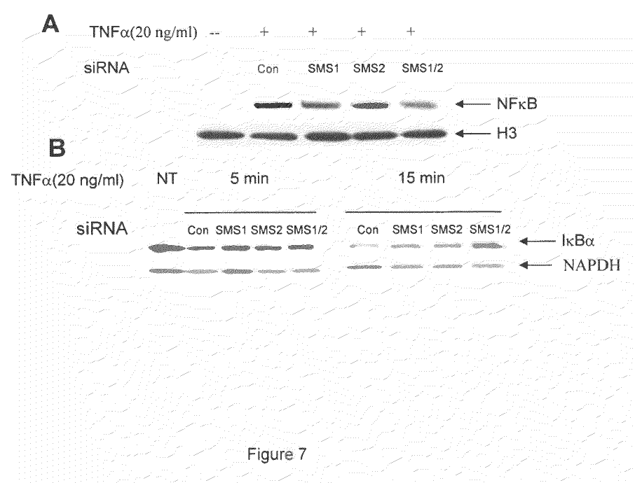

SMS1 and SMS2 expression positively correlate with levels of SM in lipid rafts.¹⁸⁻²⁰ SMS1 is involved in the regulation of lipid raft SM level and raft functions.^{18,19} In this study, it is shown that SMS1 knockdown in HEK293 cells also attenuates NFκB activation (FIG. 7-9). In HEK293 cells the expression of SMS1 and SMS2 is almost 1:1 (Hailemariam and Jiang, unpublished observation). Hence their contribution to total SMS activity and cellular SM content is proportional. In mouse macrophages, the mRNA of SMS1 to SMS2 is 4:1 (Hailemariam and Jiang, unpublished observation). As a result, SMS2 contributes to lesser proportion of the total cellular SMS activity in these cells. In either cell types because of the difference in their sub-cellular localization, each of SMS1 or SMS2 may be responsible for a local pool of cellular SM. As SMS2 is plasma membrane associated, its contribution to this pool of SM is substantial independent of its role in the total SMS activity. This is strongly suggested by the lysenin sensitivity assays in both cell types (FIG. 1G and FIG. 1H).

In conclusion, SMS2 physiologically contributes to de novo SM biosynthesis and plasma membrane SM levels, and also affects the metabolism of DAG and ceramide. Perturbations to the balance of these molecules by SMS2 inhibition caused blunted NFκB responses to inflammatory/immunological stimuli. Thus, regulation of SMS2 activity may have an important impact on inflammation, thus influence atherogenic processes.

An aspect of the present invention provides a method of screening for NFκB inhibiting agents. The method of screening NFκB inhibiting agents includes administering a biologically effective amount of a candidate SMS2 inhibitor to at least one cell; and determining whether the candidate SMS2 inhibitor inhibits NFκB.

The administration step may be done by contacting the candidate SMS2 inhibitor with one or more of the at least one cell. The candidate SMS2 inhibitor and the at least one cell may be admixed, as with a suspension, or the candidate SMS2 inhibitor may be topically applied or coated onto the at least one cell. One or more various methods of administration may be done, as may be desired.

Optionally, the administering step may further include administering the candidate SMS2 inhibitor to a mammal. The mammal subject can be one or more common laboratory experimental species, including, hamsters, guinea pigs, mice, rats, rabbits, and the like. Similarly, the mammal may be a primate, including for example a chimpanzee or a monkey. Also, the mammal may be a human subject. The administration step to a mammal may be done by injection, intravenous subcutaneous intraperitoneal, or intramuscular, and other methods of administration, as are known in the art and as may be desired.

The method further includes measuring an amount of sphingomyelin in at least one plasma membrane of each of the at least one cell, an amount of lipid rafts of each of the cells, and a combination thereof. Also, the method may further includes measuring an amount of sphingomyelin in the plasma membranes, wherein a decrease in the amount of sphingomyelin correlates to a reduction in an NFκB activation. Known methods, procedures, and assays may be used to take such measurements.

The method may further include the step of determining whether an amount of eramide has changed and/or whether an amount of diacylglycerol has changed, after the administering step. Various known methods may be employed to take measurements, analyze assays, and calculate a change, including an increase or a decrease in one or more levels as compared to a pre-administration measurement. Alternatively or in combination with comparing a previous measurement of that cellular sample or subject, one may use a standard medical text, computer correlation program, or comparative results based on known standards or tests may be used.

The method may further include the step of whether the SMS2 candidate inhibitor is an SMS2 inhibitor. This may be determined based on measurements, calculations, or observations related to at least one of ceramide levels, SM in the plasma membrane and/or lipid rafts, diacylglycerol, PC. Also, one or more of the experiments or procedures previously discussed may be likewise employed to characterize a candidate SMS2 inhibitor as a SMS2 inhibitor.

Once a successful SMS2 inhibitor is identified, the SMS2 inhibitor may be used in treating a subject having atherosclerosis with a biologically effective amount of the SMS2 inhibitor. Similarly, the SMS2 inhibitor may treat a subject having dyslipidemia, or NFκB related inflammation.

Another aspect of the present invention provides a method for attenuating inflammation induced by NF-kB. The method of attenuating inflammation induced by NF-kB further includes inhibiting sphingomyelin synthase (SMS2) in the plasma membrane of at least one cell. Inhibiting SMS2 likewise prevents activation of NF-kB, thus SMS2 may be used to prevent inflammation induced or otherwise caused by NF-kB activation. The inhibiting step may further include administering an SMS2 inhibitor to the at least one cell. The at least one cell may be in a mammal, as previously discussed.

Still another aspect of the present invention provides a method of regulating an NFκB activation which includes modulating an SMS2 in at least one cell. SMS2 may be modulated in at least one cell by genetically modulating the at least one cell. Also, SMS2 may be modulated in at least one cell by administering an SMS2 inhibiting agent that modulates the SMS2 in the at least one cell. Further, the method may include the step of reducing the SMS2 in the at least one cell, which may correlates to reducing a sphingomyelin level and an NFκB level in the at least one cell.

There is a need for a method to effectively screen Sphingomyelin synthase (SMS2) inhibitors as candidates for anti-inflammatory drugs and/or cholesterol inhibition drugs. These drug candidates may be employed in a mammal subject in order to inhibit or attenuate the NF-kB activity of the mammal, which may reduce inflammation in the mammal.

The drug candidates which may be identified may inhibit or otherwise attenuate NFκB activity, thus reducing inflammatory effects in the body of a subject. This may be used, for example, to treat diagnoses including dyslipidemia and atherosclerosis (inflammation of the arterial walls promoted by low density lipoproteins).

The SMS2 inhibitors that can be used to reduce NFκB activation, or modulate one or more NFκB related conditions, diseases, or disorders may be effective at inhibiting cholesterol absorption and/or reducing inflammation. The SMS2 inhibitors may be administered to an individual either individually or in combination with one or more known reagents, medicaments, compounds, or treatments, such that pharmaceutically acceptable delivery may result.

### EXAMPLES & METHODS

To investigate the role of SMS2 in NFκB activation macrophages from SMS2 knockout (KO) mice, and SMS2 siRNA-treated HEK 293 cells were utilized. An unexpected result was discovered, that NFκB activation and its target gene expression are attenuated in macrophages from SMS2 KO mice in response to LPS stimulation, and in SMS2 siRNA-treated HEK 293 cells after TNFalpha simulation. In line with attenuated NF-κB activation, surprisingly, SMS2 deficiency substantially diminished the abundance of toll like receptor 4 (TLR4)-MD2 complex levels on the surface of macrophages after LPS stimulation, and SMS2 siRNA treatment reduced TNFα-stimulated lipid raft recruitment of TNF receptor-1 (TNFR1) in HEK293 cells. Thus, SMS2 deficiency decreased the relative amounts of SM and diacylglycerol (DAG), and increased ceramide, suggesting multiple mechanisms for the decrease in NFκB activation.

### Nuclear and Cytoplasmic Protein Preparation

The method is previously described by Dignam.²¹ Briefly, cells were washed in cold PBS and lysed in buffer (10 mM Hepes pH 7.9, 1.5 mM MgCl₂, 10 mM KCl, 0.5 mM DTT, 0.01% NP-40) containing protease inhibitors. Nuclei were pelleted by centrifugation at 650 g for 5 minutes at 4° C. and the supernatant was collected as the cytoplasmic fraction. Nuclei were then resuspended in a buffer containing (10 mM Hepes pH 7.9, 1.5 mM MgCl₂, 10 mM KCl, and 0.5 mM DTT) and incubated on ice for 30 min with continuous agitation. The extract was recovered after centrifugation for 10 min at 12000 rpm at 4° C. Proteins were separated on SDS-PAGE gels (Bio-Rad) and Western blots were conducted with specific antibodies to p65 (NFκB) or IkBα. Anti-histone 3 (H3) and anti-GAPDH were used as nuclear and cytoplasmic control, respectively.

### Electromobility-Shift Assay (EMSA)

Nuclear extracts (6 microg) from macrophages were incubated on ice for 30 min with a [³²P]-labeled oligonucleotide comprising the proximal NFκB binding regions of the murine iNOS promoter (5′-CCAACTGGGGACTCTCCCTTTGGGAACA-3′) SEQ ID NO:1,²⁵ in 25 mM HEPES (pH 7.9), 100 mM KCl, 4% ficoll, 5 uM ZnCl2, 1 mM DTT, 0.05% NP-40, 5 mM MgCl₂, 1 ug/mL BSA and 50 ng/uL poly dI-dC in a final volume of 15 μl. Competition analysis was performed with 50-fold excess of unlabeled oligonucleotides. For supershift, samples were incubated with 2 microg of antibodies for an additional 30 min on ice. Antibodies (all from Santa Cruz) to p65, p50, p300, C-rel, and Mitf were used in supershift assay. The reaction products were separated by 5% PAGE at 4 degrees C. and visualized by autoradiography.

### SMS2 KO Mouse

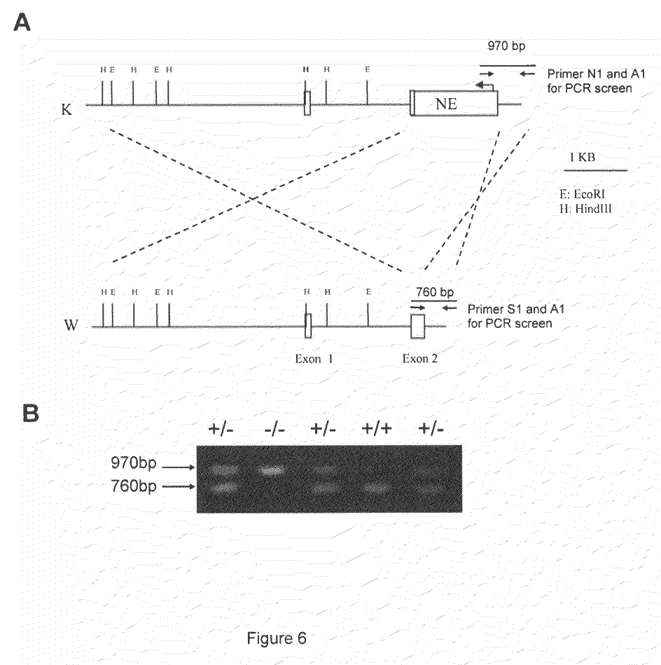

The overall strategy for gene targeting was to replace 90% of exon 2, with the neomycin-resistant gene. Because exon 2 contains the translation initiation codon ATG, deletion of exon 2 would be predicted to create an SMS2 null mouse allele (FIG. 6). The homologues recombination was screened by PCR. Sense primer N1 (5′-tgcgaggccagaggccacttgtgtagc-3′) SEQ ID NO: 2 and antisense primer A1 (5′-tgtagccctggctgttctgtactc-3′) SEQ ID NO: 3 can amplify a 970 bp fragment (KO), while, sense primer S1 (5′-cgactccaccaacacttacacaag-3′) SEQ ID NO: 4 and antisense primer A1 (5′-tgtagccctggctgttctgtactc-3′) SEQ ID NO: 5 can amplify a 760 bp fragment (wild type) (FIG. 6). The SMS2 KO mice had 129 mouse genetic background. They have backcrossed with C57BL/6 mice three generations. The animals (WT and KO) used in this study were littermates.

### Cell Culture and Transfection

Bone marrow from SMS2 KO mice was cultured for 7 days in DMEM medium supplemented with 20% L-cell medium to provide M-CSF and induce the differentiation of monocytes into macrophages. Human embryonic kidney (HEK) 293 cells were cultured in DMEM medium with 10% fetal bovine serum (FBS), 2 mM-glutamine, and 100 U/ml penicillin and streptomycin. The target sequence for SMS2 siRNA is; 5′-CCGTCATGATCACAGTTGTA-3′ SEQ ID NO: 6. For control, cells were transfected with the scrambled siRNA target sequence 5′-GAC GAC GGA GTG TGT TA ATTA-3′ SEQ ID NO: 7. The siRNA was diluted in Opti-MEM (Invitrogen) medium and transfected into cells grown to 50-70% confluence, using Lipofectamine2000 (Invitrogen).

### Cell Surface Receptor Analysis by FACS

HEK293 cells and macrophages were stained with 1 μg/mL TNFR1 antibody (PE), and with 1 ug/mL TLR4/MD-2 complex antibody (Stressgen), respectively, for 1 hr on ice, then washed with ice cold PBS 3 times before analyzed on a FACScan with CellQuest software (Becton Dickinson).

### TNFα Binding and TNFR1 Internalization Assay

Cells transfected with control or SMS2 siRNA were incubated with DMEM medium and 1.5 ng of [¹²⁵I]-labeled human recombinant TNFα (specific activity, 1.11 MBq/μg; NEN Life Sciences) for 1 hr at 4° C. for binding assay or at 37° C. for internalization. For competition binding assay a 100-fold excess of unlabeled human recombinant TNFα was used. At the end of incubation cells were washed three times with cold PBS and radioactivity was measured on a γ-counter. The specific binding is determined by subtracting the competitive binding from total binding. The amount of internalized receptor is determined as the difference between whole-cell associated radioactivity and the specific binding.

### Lipid Analyses by LC MS/MS

Ceramides comprised of a D-erythro-sphingosine backbone and a fatty acid amide were determined by a 2D LC-ESI MS/MS method. Lipid extracts from cells were injected onto a normal-phase column, where the polar lipids were retained, while the ceramide fractions were trapped on a reversed-phase column. Ceramides were eluted, separated, and detected using a triple quadruple mass spectrometer equipped with positive ion electrospray ionization (ESI) and selected reaction monitoring. Levels of PC and SM were measured via a flow injection ESI-MS/MS method. Protonated molecular ions of PC/SM species are selected by precursor ion scans of m/z 184 and the ion intensities across the flow injection profile were summed together, and after isotope correction, the quantities of each PC and SM species are then calculated relative to PC and SM internal standards.

mRNA Analyses

RNA was isolated from cells using TriZol (Invitrogen). The mouse primers used for SMS2 RT-PCR were: Forward 5′-GGTTCCCACAGAAACCAAGA-3′ SEQ ID NO: 8, and reverse; 5′-GATGCCTGTTTTCCACCACT-3′ SEQ ID NO: 9. For HEK293 cells, SMS2 mRNA was determined by real-time polymerase chain reaction (PCR) using Taqman® Gene Expression Assay (Applied Biosystems, assay ID Hs00380453_m1). 18S rRNA was used as internal control. The forward and reverse primer sequences for 18S rRNA are: 5′-AGTCCCTGCCCTTTGTACACA-3′ SEQ ID NO: 10 and 5′-GATCCGAGGGCCTCACTAAAC-3′ SEQ ID NO: 11, respectively, and the probe sequence is 5′-CGCCCGTCGCTACTACCGATTGGT-3′ SEQ ID NO: 12. The Sybergreen (SuperArray) method was used for iNOS mRNA determination; forward primer sequence: 5′ GTC TTG CAA GCT GAT GGT CA 3′ SEQ ID NO: 13; and reverse primer sequence: 5′ ACC ACT CGT ACT TGG GAT GC 3′ SEQ ID NO: 14.

### SMS Activity Assay

Cells were homogenized in a buffer containing 50 mM Tris-HCl, 1 mM EDTA, 5% sucrose, and a cocktail of protease inhibitors (Sigma). The homogenate was centrifuged at 5000 rpm for 10 minutes and the supernatant was mixed in assay buffer containing 50 mM Tris-HCl (pH 7.4), 25 mM KCl, C₆—NBD-ceramide (0.1 μg/μl), and phosphotidylcholine (0.01 μg/l). The mixture was incubated at 37° C. for 2 hours. Lipids were extracted in chloroform:methanol (2:1), dried under N₂ gas, and separated by thin layer chromatography (TLC). For de novo biosynthesis assay, cells were incubated in DMEM and 10% FBS together with [¹⁴C]-L-serine (0.2 μci/ml), substrate for SM biosynthesis. After 2-hr incubation, cellular lipids were extracted as above, separated on TLC and scanned with a Phosphoimager. Band intensity was quantified by Image-Pro Plus 4.5 (Media Cybernetics Inc.).

### Lysenin Treatment and Cell Mortality Measurement

Cells were washed twice in PBS and incubated with lysenin, 50 ng/ml 1 hr for HEK 293 and 200 ng/ml 2 hr for macrophages. Cell viability was measured using the WST-1 cell proliferation reagent according to the manufacturer's instructions (Roche).

### Luciferase Assay

Overnight siRNA transfected HEK293 cells were re-transfected with a 500 ng/ml kb-luciferase construct and a 25 ng/ml renilla construct simultaneously. After 24 hr incubation in normal medium, the cells were serum starved for 2 hr, and then treated with 20 ng/ml TNFα for 8 hr. Then cells were lysed in passive lysis buffer, and used in the dual luciferase assay system according to the manufacturers protocol (Promega). Luciferase counts were standardized using the renilla values.

### Immunocytochemistry

Macrophages or HEK293 cells were grown on 1% gelatin coated cover-slips. Cells were washed twice in PBS, fixed with 4% formaldehyde rinsed again with PBS and incubated in permeabilization solution (0.1% Triton X-100, 0.1% Sodium citrate) for 5 minutes on ice. After blocking in 3% BSA in PBS at 4° C. for 1 hr, cells were incubated sequentially in an anti-NFκB antibody overnight and secondary antibody conjugated to fluorescein (Vector Laboratories) for 1 hr in the dark. They were rinsed three times in PBS, mounted with a medium containing DAPI (for nuclear staining) and visualized with a fluorescent microscope.

### Lipid Raft Isolation

Lipid raft was isolated based on insolubility in detergent and discontinuous sucrose density gradient centrifugation. Cells from two 10 cm culture dishes were lysed on ice for 30 min in 1.2 ml of 1% Triton X-100 buffer (10 mM of pH 7.4 Tris-HCl, 150 mM NaCl, 5 mM EDTA) supplemented with protease inhibitor cocktail, and homogenized with 10 strokes in glass dounce homogenizer. The homogenates (1 ml) were loaded on discontinuous (85%, 35% and 5%) sucrose gradients and centrifuged at 38,000 rpm in Beckman SW41 Ti rotor for 18 hr at 4° C. Fractions were collected and 40 μl aliquots were separated on SDS-PAGE.

### Statistical Analysis

Data is typically expressed as mean±S.D. Data between two groups were analyzed by Student's t test. A p value of less than 0.05 was considered significant.

### Results

### The Effect of SMS2 Deficiency on SMS Activity, Cellular, and Plasma Membrane SM Levels

To investigate the relationship between SMS2 and SM synthesis, gene knockout (KO) and knockdown approaches were utilized, respectively. SMS2 KO mice were established by conventional approaches (FIG. 6A). The resulting heterozygous mice were crossed, and SMS2 knockout (KO) homozygous mice were obtained (FIG. 6B). The targeted allele segregated in a Mendelian fashion. SMS2 KO mice display no obvious abnormalities, grow into adult hood, and breed normally under conventional diet and environment. As expected, SMS2 KO macrophages have no SMS2 mRNA (FIG. 1A) and have significantly reduced SMS activity (18%, P<0.05), compared with controls (FIG. 1C). Similarly, in HEK293 cells, SMS2 siRNA treatment significantly reduced SMS2 mRNA (80%, P<0.001) (FIG. 1B) and SMS activity (60%, P<0.001) (FIG. 1D), compared with control siRNA treated cells. To determine if SMS2 is involved in de novo SM biosynthesis in cells, cells were incubated with [¹⁴C]serine; a component used for SM biosynthesis, for 2 hours, and measured [¹⁴C]SM levels in total cell lipid extracts. It was found that, compared with controls, SMS2 KO macrophages and SMS2 knockdown HEK293 cells had significantly reduced intracellular [¹⁴C]SM (30% and 50%, P<0.01, respectively) (FIGS. 1E and 1F), demonstrating that SMS2 is involved in de novo SM synthesis.

Next, the cellular SM, DAG, PC, and ceramide levels were measured in SMS2 deficient cells and their controls by ESI-MS/MS. As indicated in Table 1, both SMS2 KO macrophages and SMS2 siRNA treated HEK293 cells contained significantly less SM than controls (18% and 29%, P<0.01, respectively). Interestingly, the amount of DAG, a concomitant product of SM synthesis, was significantly decreased in SMS2 KO macrophages (20%, P<0.01), but not in SMS2 siRNA HEK293 cells. The amount of ceramide was significantly increased in both SMS2 KO macrophages and SMS2 knockdown HEK293 cells (18% and 43%, P<0.01, respectively) (Table 1). There were no changes in the levels of PC. These results suggested that SMS2 activity is important in regulating cellular SM, DAG, and ceramide.

To investigate the consequences of SMS2 deficiency on plasma membrane SM levels in intact cells, the sensitivity of cells to lysenin was measured, a SM-specific cytotoxic protein.²² Lysenin recognizes and binds SM only when it forms aggregates or domains.²³ As indicated in FIGS. 1G and 1H, both SMS2 KO macrophages and SMS2 siRNA treated HEK293 cells showed significantly less sensitivity to lysenin-mediated cytolysis than their corresponding controls (P<0.01), highlighting the critical and physiological role of SMS2 in regulating SM levels in cell membrane microdomains.

### SMS2 Deficiency Attenuates NFκB Activation and NFκB Regulated Gene Expression.

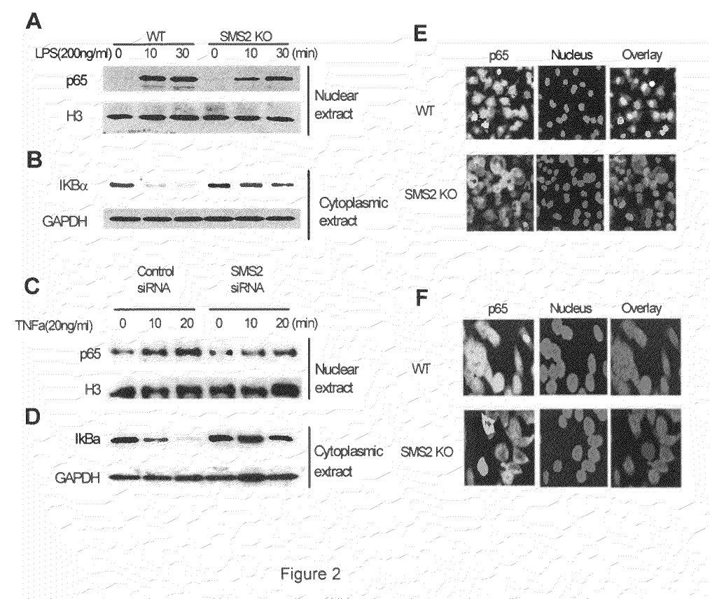

To determine the role of SMS2 in NFκB activation, ligand-induced NFκB activation was compared in SMS2 KO macrophages and SMS2 knockdown HEK293 cells with their corresponding controls. As shown in FIG. 2, SMS2 KO macrophages had decreased levels of NFκB in their nuclei compared with controls after LPS stimulation (FIG. 2A). Then, western blot was used to measure cytoplasmic IκBα, which must be degraded for NFκB to become activated, and it was found that its degradation is attenuated (FIG. 2B). These results indicated that SMS2 deficiency diminishes IκBα degradation leading to reduced nuclear translocation of NFκB.

We did similar experiments with HEK293 cells. SMS2 or control siRNA transfected cells were treated with TNFα for various time points. As shown in FIGS. 2C and 2D, SMS2 knockdown cell nuclei contain significantly less NFκB, while the cytoplasmic fraction contains significantly more IκBα, than corresponding controls. These results again indicate a linkage between SMS2 activity and NFκB activation. Also HEK293 cells were treated with SMS1 siRNA and found that SMS1 knockdown also attenuates NFκB activation (FIGS. 7A and 7B).

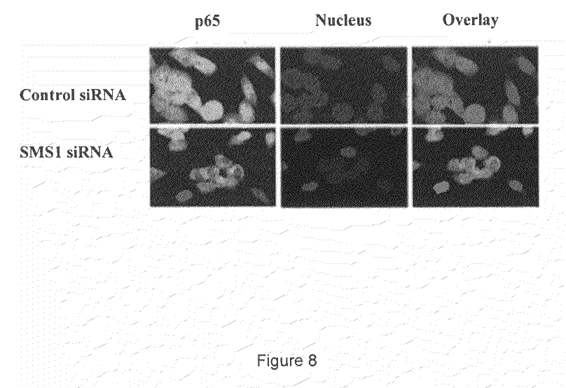

To confirm the above findings and to directly visualize the nuclear translocation of NFκB, immunocytochemistry was employed. In support of the present inventors' earlier findings, after LPS treatment, NFκB was localized in the nucleus in almost all of the wild type macrophages, while nuclear localization was greatly diminished in SMS2 KO macrophages (FIG. 2E). Similarly, in SMS2 knockdown HEK293 cells, after TNFα stimulation, the translocation of NFκB to the nucleus was also substantially reduced compared with controls (FIG. 2F). Moreover, it was found that SMS1 knockdown also attenuates NFκB activation in HEK293 cells after TNFα stimulation (FIG. 8).

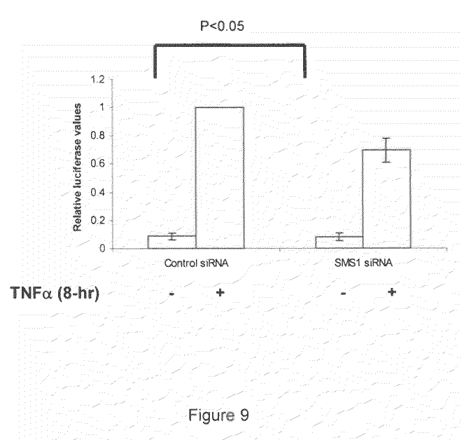

To investigate whether the inhibition of NFκB activation affects its transcriptional activity, a reporter gene assay was carried out in SMS2 knockdown HEK293 cells using a κB-luciferase plasmid. Stimulation of control siRNA treated cells with TNFα for 8 hours resulted in a nearly ten fold induction in luciferase activity (FIG. 3A) compared with untreated cells. However, in the SMS2 knockdown cells, there was a significant reduction (P<0.01) in the induction of luciferase activity (FIG. 3A). This result implies that SMS2 depletion might affect the expression of many NFκB regulated genes. It was also found that SMS1 knockdown in HEK293 cells reduced KB-luciferase expression levels (FIG. 9).

To evaluate the physiological role of NFκB attenuation caused by the SMS2 deficiency in macrophages, the LPS-induced expression of iNOS, a pro-inflammatory gene whose expression is regulated by NFκB was evaluated.²⁴ The mRNA and protein levels of iNOS in LPS-stimulated macrophages were determined by real-time PCR and western blot, respectively. As shown in FIGS. 3B and 3C, for both durations of LPS treatment, the induction in iNOS mRNA and protein levels were significantly lower in SMS2 KO than in controls, suggesting the regulation of an inflammatory process by SMS2.

To investigate whether the suppression in iNOS gene expression was due to lack of binding of NFκB to the iNOS promoter, EMSA was conducted using a native iNOS promoter fragment carrying NFκB binding sites.²⁵ NFκB binding was indicated by a supershift with antibodies to the p50 or p65 subunits and there was no supershift when three control antibodies (C-Rel, p300, and Mitf) were used (FIG. 3D). As shown in FIG. 3E, after LPS stimulation, the NFκB (p50/p65) promoter binding activity was markedly diminished in SMS2 KO macrophages compared with control. This result suggests that the reduction in iNOS transcription (FIGS. 3B and 3C) was due to the decrease in NFκB available to bind the iNOS promoter. Also, there was an unknown shifted complex with diminished NFκB binding noted in SMS2 KO macrophages (FIGS. 3D and 3E). This unknown complex could not be supershifted by p50, p65, C-Rel, p300, or Mitf antibodies (FIG. 3D).

### SMS2 Deficiency Impairs TNFR1 Recruitment to Lipid Rafts and TLR4-MD2 Complex Recruitment to Plasma Membrane

Lipid rafts play essential role in TNFR1 clustering and NFκB activation.⁷ Hence, it was investigated whether SMS2 knockdown affects TNFα mediated receptor clustering to lipid rafts. Lipid were isolated rafts based on their insolubility in 1% Triton X-100 buffer at 4° C. and centrifugation on discontinuous sucrose density gradient. Lipid rafts were found in light fractions enriched in the raft marker Src kinase lyn (FIG. 4A). The transferring receptor, CD71, is a non-raft marker. As seen on FIG. 4, before stimulation, raft regions contain a small amount of TNFR1. The recruitment of TNFR1 into raft regions was greatly increased upon TNFα stimulation in control siRNA treated cells at both time points (5 min and 15 min, FIG. 4B). However, in SMS2 knockdown cells, the recruitment of TNFR1 to the lipid rafts was greatly impaired (control siRNA vs SMS2 siRNA under raft, FIG. 4B). SMS2 siRNA did not affect total cellular TNFR1 levels (FIG. 4C). These results suggest that SMS2 deficiency-mediated SM depletion in plasma membrane lipid rafts interferes with TNFR1 clustering.

Deficiency of SMS1 has been shown to block raft-mediated internalization of ALP in mouse lymphoma cells (S49AR).¹⁹ Next, the effects of SMS2 gene knockdown on TNFα-induced TNFR1 endocytosis was investigated, as the process might be related to NFκB activation.²⁶ As shown in FIG. 5, although there are no changes in total TNFR1 on cell surface (FIG. 5A) nor in the specific binding of TNFα to surface receptor (FIG. 5B), the internalization of TNFα-TNFR1 complex, following binding, is impaired in SMS2 knockdown cells (FIG. 5C). This result provides additional evidence for the dysfunction of lipid rafts and TNFR1 as a result of SMS2 deficiency.

In macrophages, LPS-induced cell surface recruitment of TLR4 and its coreceptor MD2, a consequence of signaling upstream of NFκB activation was investigated.²⁷ FACS analysis showed that, after LPS stimulation, SMS2 KO macrophages contained fewer TLR4-MD2 complexes on the cell surface than control macrophages (FIG. 5D). This result indicates SMS2 is needed for LPS induced cell surface TLR4-MD2 complex recruitment.

It is conceivable that SMS2 deficiency should also influence signal pathways other than NFκB. To investigate this possibility, western blot for MAP kinases, p38 and p42/44, in SMS2 KO and WT macrophages after LPS stimulation was performed. It was found that both phospho-p38 and phosphor-p42/44, the active form of the kinases, are decreased in KO macrophages while total protein levels are increased (FIG. 10).

TABLE I
Lipid Concentrations (nmol/mg Protein) in SMS2-
Knockdown HEK293 cells and SMS2 KO macrophages. Mean ± SD.

| | SM | PC | Ceramide | DAG |
|---|---|---|---|---|
| Scrambled siRNA | 9.5 ± 1.1 | 90.7 ± 8.9 | 0.49 ± 0.05 | 2.72 ± 0.15 |
| SMS2 siRNA | 6.7 ± 1.1^{†} | 99.7 ± 9.8 | 0.70 ± 0.06^{†} | 2.59 ± 0.26 |
| WT | 42.1 ± 4.7 | 71.2 ± 2.9 | 1.26 ± 0.09 | 1.89 ± 0.19 |
| SMS2 KO | 34.3 ± 2.9^{†} | 74.0 ± 5.1 | 1.49 ± 0.11^{†} | 1.51 ± 0.11^{†} |

* Average of 4 experiments.
^{†}P < 0.01 by Student t test.
SMS, sphingomyelin synthase;
SM, sphingomyelin;
PC, phosphatidylcholine;
DAG, diacylglycerol.

Various changes and modifications may be made in the present invention. It is intended that all such changes and modifications come within the scope of the invention as set forth in previous discussion.

The protocols described in the application for carrying out the claimed methods are well known in the art, and are generally described in these references. All publications mentioned herein are cited for the purpose of familiarizing the reader with the background of the invention. Nothing herein is to be construed as an admission that these references are prior art in relation to the inventions described herein.
1 Libby P. Inflammation in atherosclerosis. Nature. 2002:420:868-874.
2. Mayo M W, Baldwin A S. The transcription factor NF-κB: control of oncogenesis and cancer therapy resistance. Biochim Biophys Acta. 2000; 1470:M55-62.
3. Branen L, Hovgaard L, Nitulescu M, Bengtsson E, Nilsson J, Jovinge S. Inhibition of tumor necrosis factor-{alpha} reduces atherosclerosis in apolipoprotein E knockout mice. Arterioscler Thromb Vasc Biol. 2004; 24:2137-2142.
4. Whitman S C, Ravisankar P, Daugherty A. IFN-gamma deficiency exerts gender-specific effects on atherogenesis in apolipoprotein E^{-/-} mice. J Interferon Cytokine Res. 2002; 22: 661-670.
5. Simons K, Ikonen E. Functional rafts in cell membranes. Nature. 1997; 387:569-572.
6. Simons K, Ikonen, E. How cells handle cholesterol. Science. 2000; 290:1721-1726.
7. Legler D F, Micheau O, Doucey M A, Tschopp J, Bron C. Recruitment of TNF receptor 1 to lipid rafts is essential for TNFalpha-mediated NF-κB activation. Immunity. 2003; 18:655-664.
8. Hunter I, Nixon G F. Spatial compartmentalization of tumor necrosis factor (TNF) receptor 1-dependent signaling pathways in human airway smooth muscle cells. Lipid rafts are essential for TNF-alpha-mediated activation of RhoA but dispensable for the activation of the NF-κB and MAPK pathways. J Biol Chem. 2006; 281:34705-34715.
9. Triantafilou M, Miyake K, Golenbock T, Triantafilou K. Mediators of innate immune recognition of bacteria concentrate in lipid rafts and facilitate lipopolysaccharide-induced cell activation. J. Cell. Sci. 2002; 115:2603-2611.
10. Higuchi M, Singh S, Jaffrezou J P, Aggarwal B B. Acidic sphingomyelinase-generated ceramide is needed but not sufficient for TNF-induced apoptosis and nuclear factor-κB activation. J. Immunol. 1996; 157:297-304.
11. Schütze S, Potthoff K, Machleidt T, Berkovic D, Wiegmann K, Krönke M. TNF activates NF-κB by phosphatidylcholine-specific phospholipase C-induced “acidic” sphingomyelin breakdown. Cell. 1992; 71:765-776.
12. Gamard C J, Dbaibo G S, Liu B, Obeid L M, Hannun Y A. Selective involvement of ceramide in cytokine-induced apoptosis. Ceramide inhibits phorbol ester activation of nuclear factor κB. J. Biol. Chem. 1997; 272:16474-16481.
13. Merrill A H, Jones D D. An update of the enzymology and regulation sphingomyelin metabolism. Biochim. Biophysi. Acta. 1990; 1044:1-12.
14. Luberto C, Yoo D S, Suidan H S, Bartoli G M, Hannun Y A. Differential effects of sphingomyelin hydrolysis and resynthesis on the activation of NF-κB in normal and SV40-transformed human fibroblasts. J. Biol. Chem. 2000; 275:14760-14766.
15. Schutze S, Potthoff K, Machleidt T, Berkovic D, Wiegmann K, Kronke M. TNF activates NF-κB by phosphatidylcholine-specific phospholipase C-induced “acidic” sphingomyelin breakdown. Cell. 1992; 71:765-766.
16. Huitema K, van den Dikkenberg J, Brouwers J F, Holthuis J C. Identification of a family of animal sphingomyelin synthases. EMBO J. 2004; 23:33-44.
17. Yamaoka S, Miyaji M, Kitano T, Umehara H, Okazaki T. Expression cloning of a human cDNA restoring sphingomyelin synthesis and cell growth wild type in sphingomyelin synthase-defective lymphoid cells. J. Biol. Chem. 2004; 279:18688-18693.
18. Miyaji M, Jin Z X, Yamaoka S, Amakawa R, Fukuhara S, Sato S B, Kobayashi T, Domae N, Mimori T, Bloom E T, Okazaki T, Umehara H. Role of membrane sphingomyelin and ceramide in platform formation for Fas-mediated apoptosis. J Exp Med. 2005; 202:249-259.
19. Van der Luit A H, Budde M, Zerp S, Caan W, Klarenbeek J B, Verheij M, Van Blitterswijk W J. Resistance to alkyl-lysophospholipid-induced apoptosis due to downregulated sphingomyelin synthase 1 expression with consequent sphingomyelin- and cholesterol-deficiency in lipid rafts. Biochem J. 2007; 401:541-549.
20. Li Z, Hailemariam T K, Zhou H, Li Y, Duckworth D C, Peake D A, Zhang Y, Kuo M S, Cao G, Jiang X C. Inhibition of sphingomyelin synthase (SMS) affects intracellular sphingomyelin accumulation and plasma membrane lipid organization. Biochim. Biophysi. Acta. 2007, Volume 9, September 2007; 1771:1186-1194.
21. Dignam J D, Lebovitz R M, Roeder R G. Accurate transcription initiation by RNA polymerase II in a soluble extract from isolated mammalian nuclei. Nucleic Acids. Res. 1983; 11:1475-1489.
22. Yamaji A, Sekizawa Y, Emoto K, Sakuraba H, Inoue K, Kobayashi H, Umeda M. Lysenin, a novel sphingomyelin-specific binding protein, J. Biol. Chem. 1998; 273:5300-5306.
23. Ishitsuka R, Yamaji-Hasegawa A, Makino A, Hirabayashi Y, Kobayashi T R. A lipid-specific toxin reveals heterogeneity of sphingomyelin-containing membranes. Biophys J. 2004; 86:296-307.
24. Xie Q W, Kashiwabara Y, Nathan C. Role of transcription factor NF-κB/Rel in induction of nitric oxide synthase, J Biol. Chem. 1994; 269:4705-4708.
25. Xie Q W, Whisnant R, Nathan C. Promoter of the mouse gene encoding calcium-independent nitric oxide synthase confers inducibility by interferon gamma and bacterial lipopolysaccharide. J Exp Med. 1993; 177:1779-1784.
26. Schneider-Brachert W, Tchikov V, Merkel O, Jakob M, Hallas C, Kruse M L, Groitl P, Lehn A, Hildt E, Held-Feindt J, Dobner T, Kabelitz D, Krönke M, Schütze S. Inhibition of TNF receptor 1 internalization by adenovirus 14.7K as a novel immune escape mechanism. J Clin Invest. 2006; 116:2901-2913.
27. Ogawa S, Lozach J, Benner C, Pascual G, Tangirala R K, Westin S, Hoffmann A, Subramaniam S, David M, Rosenfeld M G, Glass C K. Molecular determinants of crosstalk between nuclear receptors and toll-like receptors, Cell. 2005; 122:707-721.
28. Tafesse F G, Ternes P, Holthuis J C. The multigenic sphingomyelin synthase family. J Biol Chem. 2006; 281:29421-29425.
29. Tafesse F G, Huitema K, Hermansson M, van der Poel S, van den Dikkenberg J, Uphoff A, Somerharju P, Holthuis J C. Both sphingomyelin synthase SMS1 and SMS2 are required for sphingomyelin homeostasis and growth in human HeLa cells. J Biol. Chem. 2007; 282:17537-17547.
30. Lee N P, Mruk D D, Xia W, Cheng C Y. Cellular localization of sphingomyelin synthase 2 in the seminiferous epithelium of adult rat testes. J Endocrinol. 2007; 192:17-32.
31. Wakelam M J. Diacylglycerol—when is it an intracellular messenger? Biochim Biophys Acta. 1998; 1436:117-126.
32. Griner E M, Kazanietz M G. Protein kinase C and other diacylglycerol effectors in cancer. Nat Rev Cancer. 2007; 7:281-294.
33. Vancurova I, Miskolci V, Davidson D. NF-κB activation in tumor necrosis factor alpha-stimulated neutrophils is mediated by protein kinase C delta. Correlation to nuclear IκBalpha. J Biol Chem. 2001; 276:19746-19752.
34. Satoh A, Gukovskaya A S, Nieto J M, Cheng J H, Gukovsky I, Reeve J R Jr, Shimosegawa T, Pandol S J. PKC-delta and -epsilon regulate NF-κB activation induced by cholecystokinin and TNF-alpha in pancreatic acinar cells. Am J Physiol Gasstroin Liver Physiol. 2004; 287:G582-591.

SEQUENCE

| | |
|---|---|
| SEQ ID NO 1 | 5′-CCAACTGGGGACTCTCCCTTTGGGAACA-3 |
| SEQ ID NO 2 | (5′-tgcgaggccagaggccacttgtgtagc-3′) |
| SEQ ID NO 3 | (5′-tgtagccctggctgttctgtactc-3′) |
| SEQ ID NO 4 | (5′-cgactccaccaacacttacacaag-3′) |
| SEQ ID NO 5 | (5′-tgtagccctggctgttctgtactc-3′) |
| SEQ ID NO 6 | 5′-CCGTCATGATCACAGTTGTA-3′ |
| SEQ ID NO 7 | 5′-GAC GAC GGA GTG TGT A ATTA-3′ |
| SEQ ID NO 8 | 5′-GGTTCCCACAGAAACCAAGA-3′ |
| SEQ ID NO 9 | 5′-GATGCCTGTTTTCCACCACT-3′ |
| SEQ ID NO 10 | 5′-AGTCCCTGCCCTTTGTACACA-3′ |
| SEQ ID NO 11 | 5′-GATCCGAGGGCCTCACTAAAC-3′ |
| SEQ ID NO 12 | 5′-CGCCCGTCGCTACTACCGATTGGT-3 |
| SEQ ID NO 13 | 5′ GTC TTG CAA GCT GAT GGT CA 3′ |
| SEQ ID NO 14 | 5′ ACC ACT CGT ACT TGG GAT GC 3′ |

What is claimed is:

1. A method of screening for NFκB inhibiting agents, comprising:
administering a biologically effective amount of a candidate SMS2 inhibitor to at least one cell; and
determining whether the candidate SMS2 inhibitor inhibits NFκB.

2. The method of claim 1, wherein the administering step further includes administering the candidate SMS2 inhibitor to a mammal.

3. The method of claim 1, further wherein the method further comprises measuring an amount of sphingomyelin in at least one plasma membrane of each of said at least one cell, an amount of lipid rafts of each of said cells, and a combination thereof.

4. The method of claim 1, further wherein the method further includes measuring an amount of sphingomyelin in said plasma membranes, wherein a decrease in said amount of sphingomyelin correlates to a reduction in an NFκB activation.

5. The method of claim 1, further wherein the method further includes determining whether an amount of ceramide has changed after said administering step.

6. The method of claim 1, further wherein the method further includes determining whether an amount of diacylglycerol has changed after said administering step.

7. The method of claim 1, further comprising the step of determining whether said SMS2 candidate inhibitor is an SMS2 inhibitor.

8. The method of claim 7, further comprising treating a subject having atherosclerosis with a biologically effective amount of said SMS2 inhibitor.

9. A method for attenuating inflammation induced by NF-kB, comprising inhibiting sphingomyelin synthase (SMS2) in the plasma membrane of at least one cell.

10. The method of claim 9, wherein the inhibiting step further comprises administering an SMS2 inhibitor to said at least one cell.

11. The method of claim 9, wherein the at least one cell is in a mammal.

12. A method of regulating an NFκB activation comprising modulating an SMS2 in at least one cell.

13. The method of claim 12, further wherein the modulating step comprises genetically modulating the SMS2 in the at least one cell.

14. The method of claim 12, further wherein the modulating step comprises administering an SMS2 inhibiting agent that modulates the SMS2 in the at least one cell.

15. The method of claim 12, further wherein reducing the SMS2 in the at least one cell correlates to reducing a sphingomyelin level and an NFκB level in the at least one cell.
